(19) United States
(12) Patent Application Publication
(10) Pub. No.: US 2010/0089896 A1
(43) Pub. Date: Apr. 15, 2010

(54) HEATABLE WRAPPING COCOON

(76) Inventor: Harvey T. Bart, New York, NY (US)

Correspondence Address: HOFFMANN & BARON, LLP, 6900 JERICHO TURNPIKE, SYOSSET, NY, 11791, US

(21) Appl. No.: 12/249,441

(22) Filed: Oct. 10, 2008

(51) Int. Cl. H05B 3/00; A61F 7/00

(52) U.S. Cl. 219/211; 219/212; 607/108; 607/109; 607/96

(57) ABSTRACT

A heatable cocoon including a body portion having an upper end and a pair of opposed side edges. The body portion is foldable to permit the pair of side edges to be folded over one another to form an enclosed interior. The body portion is formed of a first layer and second layer of supple material. A head covering portion having a concave opening extends from the upper end of the body portion. A securement device secures the opposed side edges of the body portion together. A plurality of enclosures is formed between the first and second layers. A plurality of heating devices is provided with one heating device of the plurality being disposed and sealed within each of the plurality of enclosures.

### FIELD OF THE INVENTION

The present invention relates to a wrapping device and more particularly a cocoon having heat generating elements, the cocoon covering one's body and head.

### BACKGROUND OF THE INVENTION

Blankets and clothing such as gloves, socks and coats have been made including devices which generate heat in order to keep a wearer comfortable. Traditionally, such devices use heating coils located within the fabric connected to a power supply, such as batteries. Accordingly, the devices do not work by simply insolating the wearer from the ambient conditions to maintain natural body heat, but also has an independent heating element to provide additional warmth. The effectiveness of such devices, however, relies on the power supply being available and active when the need for the warming device arises. Such devices are not suitable for long term storage since the batteries tend to discharge over time.

Pocket warmers using fuel, such as lighter fluid or naphtha, to create a catalytic reaction are also well known in the art and have been placed within the pockets of garments in order to keep a wearer warm. Such warmers need to be refueled, and it can be difficult to both start and maintain the catalytic reaction.

Heating devices relying on slow and sustained chemical reactions in order to generate heat are also well known in the art. Such devices include exothermic packs which are activated upon exposure to the atmosphere. The exposure to the oxygen in the atmosphere creates a chemical reaction with the contents of the packs, generating heat for a certain amount of time. Once the chemical reaction runs its course, the heat is no longer generated and the pack can be disposed. Such exothermic heating packs are disclosed, for example, in U.S. Pat. No. 5,342,412.

Disposable air-activated heating packs have been placed within blankets for providing warmth to the user. However, such blankets are designed to be wrapped around a user's body and are not configured to cover the user's head. Covering of one's head is very important to maintain body heat, since a significant amount of body heat is lost through one's head. Blankets may not be effective for providing adequate warmth especially when the user cannot hold the blanket over their head. This is the case for a small child or infant.

Accordingly, it would be desirable to provide a wrapping device having a covering for a user's body and head which has heating devices integrally formed therewith such that wrapping device the is ready to be used when needed.

### SUMMARY OF THE INVENTION

According to aspects illustrated herein, there is provided a heatable cocoon including a body portion having an upper end and a pair of opposed side edges. The body portion is foldable to permit the pair of side edges to be folded over one another to form an enclosed interior. The body portion is formed of a first layer and second layer of supple material. A head covering portion having recess, and it extends from the upper end of the body portion. A securement device secures the opposed side edges of the body portion together. A plurality of enclosures is formed between the first and second layers. A plurality of heating devices is provided with one heating device of the plurality being disposed and sealed within each of the plurality of enclosures.

According to other aspects illustrated herein, there is provided a single-use heatable infant cocoon including a body portion having a pair of opposed side edges. The body portion is foldable to permit the pair of side edges to be folded over one another to form an enclosed interior for surrounding the infant. A contoured portion extends from the body portion forming a head covering portion adapted to receive the head of an infant. The body portion is formed of a first layer and a second layer of supple material. The securement device secures the opposed side edges of the body portion together. A plurality of sealed enclosures is formed between the first and second layers. A plurality of air-activated heating devices is provided with one heating device of the plurality being disposed within each of the plurality of enclosures.

According to still other aspects illustrated herein, there is provided a disposable heatable cocoon system including a cocoon having a body portion. The body portion includes an upper end. A head covering portion having a recess for receiving a head extends from the upper end. The body portion includes a pair of opposed side edges. The body portion is foldable to permit the pair of side edges to be folded over one another to form an enclosed interior. A securement device secures the opposed side edges of the body portion together. The body portion and head portion are formed of a first layer and second layer of supple material. A plurality of enclosures is formed between the first and second layers. The plurality of enclosures each includes an air-activated heat device disposed within. An environmental barrier surrounds the cocoon, and removal of the barrier activates the heating devices.

According to further aspects illustrated herein, there is provided a method of warming a mammal including:

placing a mammal in a cocoon, the cocoon including
(i) a body portion having a pair of opposed side edges, the body portion being foldable to permit the pair of side edges to be folded over one another to form an enclosed interior for surrounding the mammal, and a contoured portion forming a head covering portion extending from the body portion for receiving the head of a mammal;
(ii) a securement device for securing opposed side edges of the body portion together;
(iii) the body portion being formed of a first layer and second layer of supple material;
(iv) a plurality of enclosures formed between the first and second layers; and
(v) the plurality of enclosures including an air-activated heat device sealed within the enclosure; and
(vi) an environmental protective barrier surrounding the cocoon, wherein removal of the barrier activates the heating devices;

placing the head covering portion over the mammal's head; and

securing the body portion around the mammal.

According to yet further aspects illustrated herein, there is provided a method of making a single-use heatable cocoon including:

forming a first layer of material to a predetermined shape;

forming a second layer of material to a predetermined shape, the first and second layers having a pair of flaps extending therefrom;

overlying the first layer with the second layer;

joining together the pair or flaps to form a head covering portion having a recess;

forming an array of enclosures between the first and second layers;

placing an exothermic heating device in each enclosure;

joining the first and second layers together to seal the enclosures and retain the heating devices therein.

### BRIEF DESCRIPTION OF THE DRAWINGS

FIG. 1 is a perspective view of a cocoon of the present invention.

FIG. 2 is a top plan view of the cocoon of FIG. 1.

FIG. 3 is a cross-sectional view of the cocoon taken along line 3-3 of FIG. 2.

FIG. 4 is a top plan view of the cocoon in a pre-finished stage.

FIG. 5 is a perspective view of a system of the present invention.

### DETAILED DESCRIPTION OF THE PREFERRED EMBODIMENTS

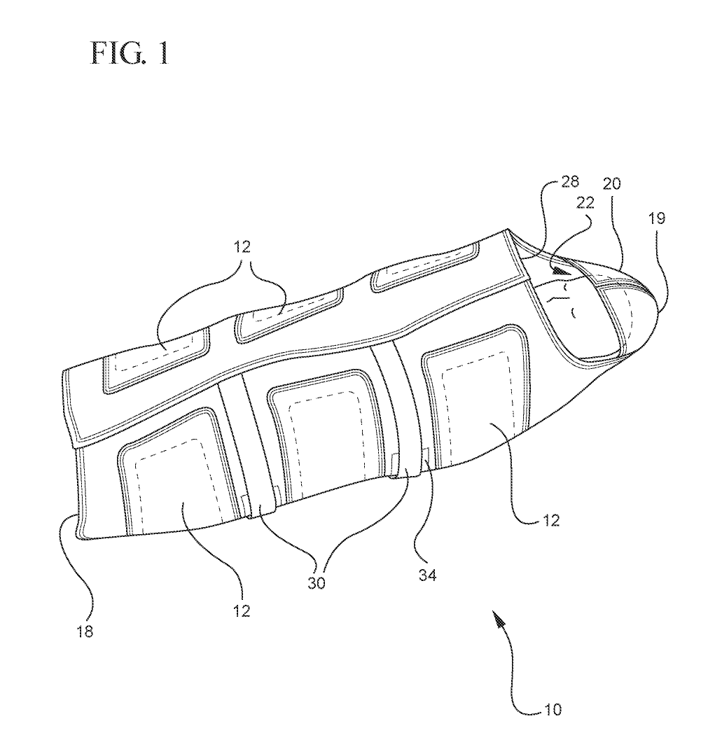

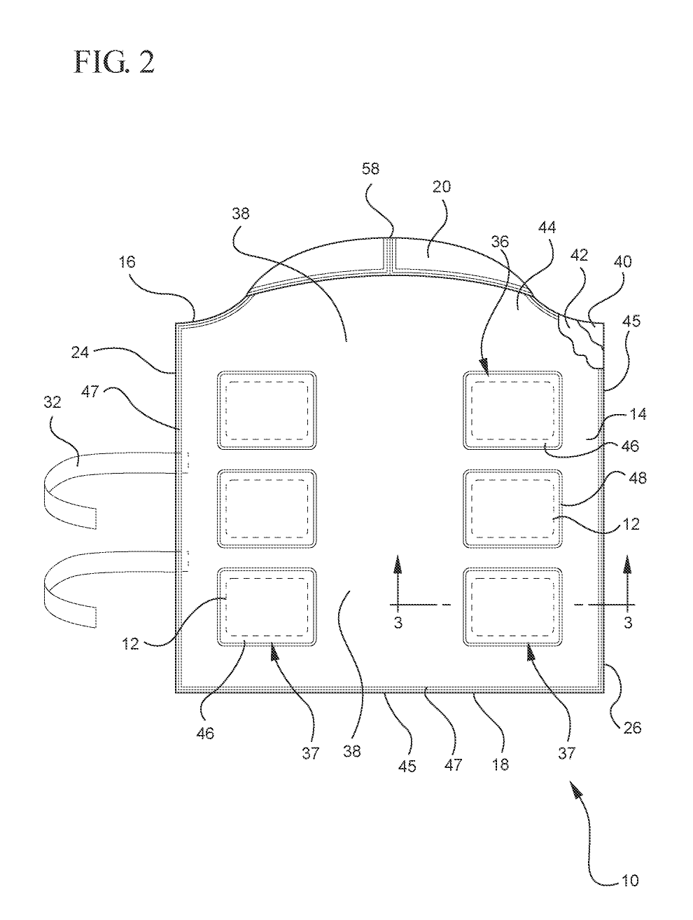

With reference to FIGS. 1 and 2, the present invention provides a cocoon 10 which wraps around the head and body of an individual. The cocoon 10 includes one or more heating devices 12 disposed thereon to provide warmth to the user. The cocoon 10 provides a covering and a source of heat which protects the user from the cold. The cocoon 10 may include a supple panel forming a body portion 14 which may be wrapped around the body of the user. The body portion includes an upper 16 and lower 18 end. A head covering portion 20 in the form of a hood which extends from the upper end 16 of the body portion 14. The head covering portion 20 is preferably integrally formed with the body portion 14. The head covering portion 20 may be contoured and may have a generally concave recess 22 for receiving a user's head. Body portion 14 may also include opposed side edges 24 and 26 which may be brought together and overlapped to form a cocoon interior 28 as shown in FIG. 1. When the cocoon 10 is closed around a user, their body and head are covered and protected from the ambient temperature.

Cocoon 10 may further include a securement device 30 including a pair of straps 32 which extend from one side edge 24 and may be secured by a fastening device 34 such as a hook and loop fastener. The securement device 30 may include other devices such as snaps, hooks, buckles, or any other type of fastening device well known in the art. The securement device 30 may be adjustable to accommodate the size of the user so that the body portion 14 may be held closely wrapped them.

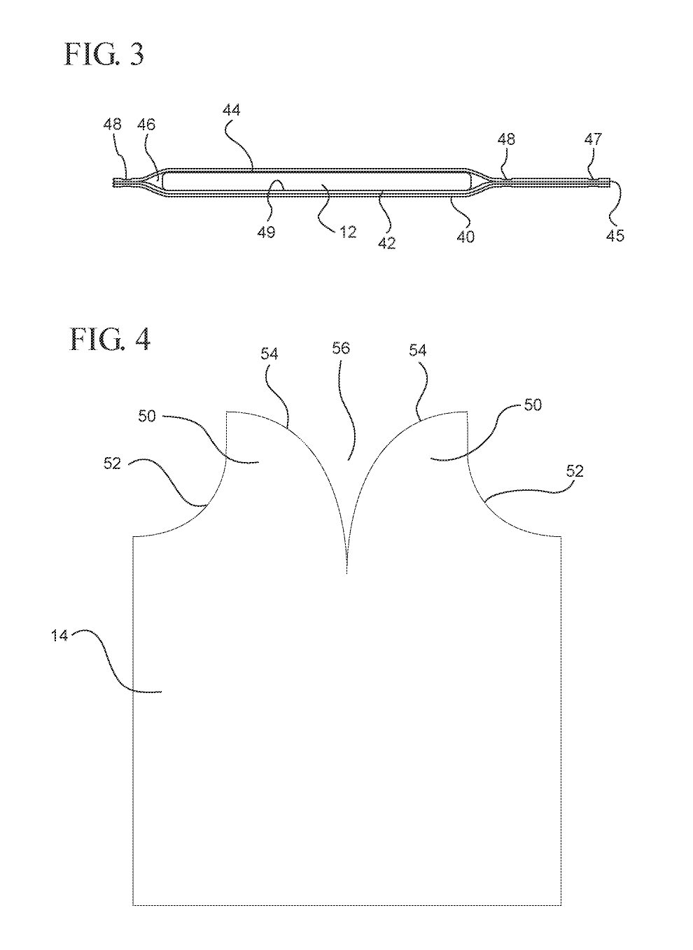

With additional reference to FIG. 3, the heating devices 12 may be supported on the body portion 14. The heating devices 12 may be disposed in an array 36 forming two columns 37 extending from the upper end 16 to the lower end 18 as shown in FIG. 2. A space 38 without heating devices separates the two columns. When the opposed side edges 24 and 26 of the body portion 14 are brought together, the heating device wraps around the side of the user providing an even, warming effect. It is within the contemplation of the present invention that the heating devices 12 may be disposed in different patterns on the cocoon as desired.

The heating devices 12 may include devices which generate heat and which may have incorporated therewith the source of energy for producing heat. In a preferred embodiment, heating device 12 may include an air-activated device wherein exposure to the air causes an exothermic reaction generating heat. Such devices are well known in the art and typically include components such as iron oxide, water and salt. In a preferred embodiment heating device may include a packet comprised of a face side-nylon non-woven fabric inside-polyethylene laminate film pierced needle holes. Disposed within the packet is iron powder (50-60%), water (15-25%), activated charcoal (5-10%), water vermiculite (1-3%), salt (2-5%), wood powder (2-5%) and water absorptive powder (0.1-1%). When exposed to the oxygen in the air, the iron oxidizes resulting in a generation of heat. The period of heat generation may be approximately 6 to 8 hours, for example. The heating device 12 may be a single-use type device wherein once it is activated and the reaction is completed and heat is no longer generated, it may be disposed of.

Alternatively, the heating devices 12 may include other types of heat generating devices that may be either chemically or electrically powered.

With further reference to FIG. 3, the cocoon 10 may be formed from multiple layers of supple material secured together. An outer layer 40 which faces away from a user's body may include a non-woven fabric which is trimmed to the proper size. The fabric may include a nylon spundbond material consisting of 98% polycapronamide and 2% titanium dioxide. It is within the contemplation of the present invention that other materials including woven materials may be used for the various layers. A second intermediate layer 42 may include an insulating layer such as a thin foam material. An inner layer 44 which faces and lies against the user may be formed of a material the same or similar to the outer layer, namely a non-woven material. The inner and outer layers permit the transmission of oxygen. The insulating intermediate layer 42 may have an insulating property greater than that of the inner 44 and outer 40 layers.

The various layers which form the cocoon 10 may be cut from a roll of material such that the layers have a similar profile. The layers may then be stacked up any overlie each other with the intermediate layer 42 disposed over the inner layer 44 and the outer layer 40 disposed on the intermediate layer 42. The edges 45 of the layers may be secured together by edge seams 47 to form the cocoon 10. The securement of the layers may be achieved, for example, by heat welding the edges together such as by radio frequency or ultrasonic welding, or by use of an adhesive, or by stitching the layers.

The heating devices 12 are preferably non-removably secured to the cocoon 10. Accordingly, the cocoon 10, along with its heating devices 12, which have the energy to produce heat, form a unitary structure. With reference to FIG. 3, in the preferred embodiment, the heating devices 12 are contained within enclosures 46 formed on the cocoon 10. The enclosures 46 may be formed by the layers which make up the cocoon 10. The heating devices 12 are sandwiched between the inner layer 44 and the insulating intermediate layer 42. An enclosure 46 may be created by forming a seam 48 entirely surrounding the heating device 10. Seam 48 may be formed in a manner similar to that used to forming seams 47, for example, by radio frequency welding. The heating devices 12 are therefore permanently sealed within the cocoon and cannot be separated therefrom. An adhesive layer 49 may be provided between the heating devices 12 and the intermediate layer 42 to secure the heating devices to the intermediate layer 42 and maintain each heating device's position within the enclosure 46.

With reference to FIG. 4, the head covering portion 20 may be formed integrally with the body portion 14 and formed from the same layers as the body portion. In a preferred embodiment, the layers 40, 42 and 44 which form the cocoon 10 may be formed having a similar shape with each having a pair of flaps 50 extending therefrom. The flaps 50 have outer side edges 52 which have a concave-shape. The flaps inner edges 54 have a convex shape. The two flaps 50 form a generally V-shaped opening 56 between them. In order to form the head covering portion 20, the inner edges 54 of the two flaps may be brought together and joined forming a head covering portion seam 58 (FIG. 2.) The head covering portion seam 58 may be formed in a manner similar to forming of seam 47. The joining of the two flaps 50 creates the head covering portion 20 and the pouch 22 thereof. Accordingly, the head covering portion 20 is formed from the same pieces of material as the body portion 14.

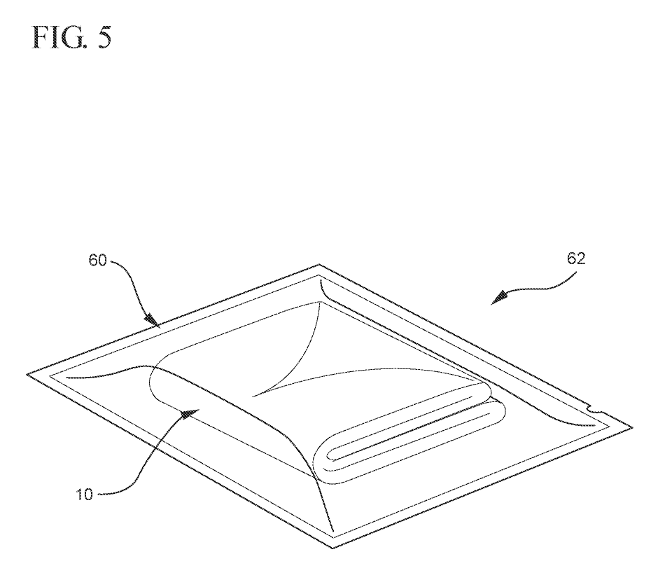

With reference to FIG. 5, the cocoon 10 may be folded and inserted within an environmental protective barrier 60 which surrounds the cocoon 10 forming a ready-to-use package 62. This barrier 60 may include a bag formed of a material which does not permit air and especially oxygen to pass therethrough. In a preferred embodiment, the barrier may be formed of a polypropylene film of vinylidene chloride coated material which has low vapor and gas permeability. The barrier 60 may be evacuated and hermetically sealed such that the cocoon 10 and the heating devices 12 are isolated from the air. In this state, the heating devices 12 are unused and not activated. The cocoon 10 may remain in this ready state until needed by a wearer.

In one embodiment of the present invention, the cocoon 10 is sized to cover a child such as an infant. Situations may occur when a child is unexpectedly exposed to a cold environment such as during a car accident or vehicle break down. In addition, an injured child may need to be kept warm to prevent the onset of shock or hypothermia. When the need arises to provide additional warmth to a child, the protective barrier 60 is opened and the cocoon 10 is removed therefrom. By exposing the cocoon 10 to air, the heating devices 12 are activated and begin to generate heat. The cocoon 10 may be unfolded to the open position shown in FIG. 2. The child may be placed on the body portion 14 and their head placed within the recess 22 of the head covering portion 20. The head, in addition to the body, is therefore covered and protected from the elements. If the child's legs do not extend to or beyond the lower end 18 of the body portion, the lower end 18 may be folded over the lower extremities. The sides of the body portion 14 are then brought together to surround the child as shown in FIG. 1. The straps 32 may then be secured to the fastening devices 34 to maintain the cocoon 10 in a closed position. With the heating devices 12 exposed to air they will begin to generate and provide heat for a finite period of time.

It is within the contemplation of the present invention that the cocoon 10 may be of a type used by humans, both children and adults; however, the cocoon could also be configured to be placed on animals where extra warmth is desired. The size of the cocoon 10 may be varied to accommodate the intended user.

Once the heating devices 12 are spent and are no longer generating heat, the cocoon 10 may be removed and dispose of. Another package 62 may be maintained by the user in case the need again arises. Upon opening the package 62, the user is present with a wrapping device that covers the body and head and a fresh set of heating devices 12 every time.

It will be appreciated that various of the above-disclosed and other features and functions, or alternatives thereof, may be desirably combined into many other different systems or applications. Various presently unforeseen or unanticipated alternatives, modifications, variations, or improvements therein may be subsequently made by those skilled in the art which are also intended to be encompassed by the following claims.

What is claimed is:

1. A heatable cocoon comprising:
a body portion including an upper end and a pair of opposed side edges, the body portion being foldable to permit the pair of side edges to be folded over one another to form an enclosed interior, the body portion being formed of a first layer and second layer of supple material;
a head covering portion having a recess, the head covering portion extending from the upper end of the body portion;
a securement device for securing the opposed side edges of the body portion together;
a plurality of enclosures formed between the first and second layers; and
a plurality of heating devices with one heating device of the plurality being disposed and sealed within each of the plurality of enclosures.

2. The cocoon as defined in claim 1, wherein the first layer forms an outer layer and the second layer forms an inner layer adapted to face toward the enclosed interior.

3. The cocoon as defined in claim 2, further including an intermediate layer disposed between the inner and outer layers.

4. The cocoon as defined in claim 3, wherein the intermediate layer is an insulating layer.

5. The cocoon as defined in claim 4, wherein the intermediate layer is formed of a foam material.

6. The cocoon as defined in claim 4, wherein the heating devices are air-activated and disposed between the inner layer and the intermediate layer.

7. The cocoon as defined in claim 1, wherein the heating devices include a source of energy for generating heat, and a seam is formed around each of the plurality of enclosures to prevent removal of the heating devices therein.

8. The cocoon as defined in claim 1, wherein the first and second layers are formed of a non-woven material.

9. The cocoon as defined in claim 1, wherein the head covering portion is integrally formed with the body portion.

10. A single-use heatable infant cocoon comprising:
a body portion including a pair of opposed side edges, the body portion being foldable to permit the pair of side edges to be folded over one another to form an enclosed interior for surrounding the infant;
a contoured portion extending from the body portion forming a head covering portion adapted to receive the head of an infant;
the body portion being formed of a first layer and a second layer of supple material;
a securement device for securing opposed side edges of the body portion together;
a plurality of sealed enclosures formed between the first and second layers; and
a plurality of air-activated heating devices with one heating device of the plurality being disposed within each of the plurality of enclosures.

11. The cocoon as defined in claim 10, wherein at least one of the first and second layers of material permit the transmission of oxygen.

12. The cocoon as defined in claim 10, wherein the heating devices are non-removably secured in the enclosures.

13. The cocoon as defined in claim 10, wherein an intermediate insulating layer is disposed between the first and second layers.

14. The cocoon as defined in claim 13, wherein the second layer for an inside surface of the cocoon and the heating devices are disposed between the insulating layer and the second layer.

15. The cocoon as defined in claim 11, wherein plurality of heating devices are arranged in an array extending between an upper end of the body portion to a lower end of the body portion.

16. The cocoon as defined in claim 15, wherein the array includes a pair of columns separated by a space.

17. A disposable heatable cocoon system comprising:
a cocoon including a body portion, the body portion including an upper end, a head covering portion having a recess for receiving a head extends from the upper end, the body portion including a pair of opposed side edges, the body portion being foldable to permit the pair of side edges to be folded over one another to form an enclosed interior;
a securement device for securing opposed side edges of the body portion together;
the body portion and head covering portion being formed of a first layer and second layer of supple material;
a plurality of enclosures formed between the first and second layers; and
the plurality of enclosures each including an air-activated heating device disposed therein; and
an environmental barrier surrounding the cocoon, wherein removal of the barrier activates the heating devices.

18. The system as defined in claim 17, wherein the environmental barrier retards the transmission of oxygen.

19. The system as defined in claim 17, wherein the environmental barrier includes a bag which is hermetically sealed.

20. The system as defined in claim 17, wherein the first and second layers are formed of a non-woven material.

21. The system as defined in claim 17, wherein the heating devices are non-removably secured within the enclosures

22. A method of warming a mammal comprising:
placing a mammal in a cocoon, the cocoon including
(i) a body portion having a pair of opposed side edges, the body portion being foldable to permit the pair of side edges to be folded over one another to form an enclosed interior for surrounding the mammal, and a contoured portion forming a head covering portion extending from the body portion for receiving the head of a mammal;
(ii) a securement device for securing opposed side edges of the body portion together;
(iii) the body portion being formed of a first layer and second layer of supple material;
(iv) a plurality of enclosures formed between the first and second layers; and
(v) the plurality of enclosures including an air-activated heat device sealed within the enclosure; and
(vi) an environmental protective barrier surrounding the cocoon, wherein removal of the barrier activates the heating devices;
placing the head covering portion over the mammal's head; and
securing the body portion around the mammal.

23. The method as defined in claim 22, including folding the body portion over the mammal and securing the securement device.

24. The method as defined in claim 22, including folding a lower end of the body portion over lower extremities of the mammal.

25. The method as defined in claim 23, including disposing of the cocoon after the heating elements have been spent.

26. The method as defined in claim 22, including opening the protective barrier thereby activating the heating devices.

27. A method of making a single-use heatable cocoon comprising:
forming a first layer of material to a predetermined shape;
forming a second layer of material to the predetermined shape, the first and second layers having a pair of flaps extending therefrom;
overlying the first layer with the second layer;
joining together the pair or flaps to form a head covering portion having a recess;
forming an array of enclosures between the first and second layers;
placing an exothermic heating device in each enclosure;
joining the first and second layer together to seal the enclosures and retain the heating devices therein.

28. The method as defined in claim 27, wherein joining the first and second layer together to seal the enclosures includes radio frequency welding the first and second layer.

29. The method as defined in claim 27, including disposing an insulating layer between the first and second layer and disposing the heating devices between the insulating layer and the second layer.

30. The method as defined in claim 29, wherein the first and second layers are formed of the same material, and the insulating layer is formed of a different material having an insulating property greater then that of the first and second layers.
